(19) United States
(12) Patent Application Publication
(10) Pub. No.: US 2018/0250079 A1
(43) Pub. Date: Sep. 6, 2018

(54) HANDPIECE REGISTER

(71) Applicant: EPED Inc., Kaohsiung City (TW)

(72) Inventors: Ta-Ko Huang, Kaohsiung City (TW); Jerry T. Huang, Kaohsiung City (TW)

(21) Appl. No.: 15/907,904

(22) Filed: Feb. 28, 2018

(30) Foreign Application Priority Data
Mar. 1, 2017 (CN) 106106706

(51) Int. Cl. A61B 34/20

(52) U.S. Cl. CPC A61B 34/20; A61B 5/061; A61B 2034/2055

(57) ABSTRACT

The present invention provides a handpiece register for positioning an instrument in a medical surgery. The handpiece register comprises a plate body, more than three optical measurement units and a positioning block. Therefore, the handpiece register of the present invention may accomplish relative position of the instrument to assist a doctor to achieve an immediate relative position of the instrument and a surgical site in the surgery.

### (a) TECHNICAL FIELD OF THE INVENTION

The present invention relates to a handpiece register, belonging to a technical field of medical devices, and more particularly to a handpiece register for positioning an instrument through a computer instantly.

### (b) DESCRIPTION OF THE PRIOR ART

With the development of medical technology, many diseases have been treated with minimally invasive surgery that is minimally destructive or minimally invasive. Currently, the micro-tools are used in the surgery, whether it is common dental implants or major surgery cardiac catheterization surgery.

Currently, an X-ray film is used for capturing the three-dimensional data of the affected area in the surgery, and a simulation is performed by the three-dimensional data to infer a relative position, which the instrument (portable instrument, portable handpiece or handpiece) needs to be moved, to assist doctors in surgery. However, it is an inevitable situation that the different instruments or the different positioning data could result in incorrect data when the operational data is used in the actual operation of the surgery. Therefore, the doctor can only depend on his own experience and technical judgment to adjust the instruments to achieve the most successful surgical results.

In order to prevent misjudgment during above process of the surgery, the present invention provides a handpiece register for immediate feedback of relative position of surgical instruments. Therefore, the position data of the front end of the instruments can be transformed into the computer software to facilitate immediate positioning information feedback to the doctors so as to reduce and prevent the risk of operation deviation or failure because of misjudgment of position in the surgery.

### SUMMARY OF THE INVENTION

To overcome the above problems, the present invention provides a handpiece register. The handpiece register may provide an immediate feedback of relative position of the instruments in a surgery to facilitate judgment and immediately adjust the instruments by a doctor, so as to reduce and prevent the risk of surgical deviation or failure because of misjudgment of position in the surgery.

To solve the above problems, the inventor of the present application provides a handpiece register, and the handpiece register is used for positioning an instrument in a medical surgery. The handpiece register comprises a plate body, more than three optical measurement units and a positioning block. The optical measurement units are all disposed on the same plane of the plate body; the positioning block is also disposed on the plate body, and the positioning block and the three optical measurement units are located on the same plane of the plate body. Further, at least one positioning base is disposed on the positioning block and is used for abutting a front end of the instrument. The front end of the instrument is abutted the positioning base, and the instrument and the positioning base are both located in a camera coverage of an image capture device. According to the optical measurement units disposed on the plate body and the optical measurement units disposed on the instrument, a computer electrically connected to the image capture device may perform a calculation to simulate an image and a position of the instrument so as to display on a screen.

Further, the plate body is a polygon with more than three sides, the plate body may also be a polygon plate body, such as a tetragon plate body, a pentagon plate body, a hexagon plate body, a polygon plate body and so on or a regular polygon plate body.

Furthermore, the optical measurement units may be an active light emitting component or a light reflecting component.

Additionally, when the optical measurement units are disposed on the instrument, the optical measurement units may be not only disposed on the instrument in a fixing method but also disposed on the instrument in an adhesive method. Therefore, the optical measurement units may be widely used on various instruments to apply to more kinds of surgeries.

In addition, the positioning block is used for abutting the front end of instrument which is provided for positioning. The instrument is abutted to a fixing position, namely, the positioning base on the positioning block, and the positioning block is a removable replacement component. Therefore, the positioning bases with different shapes may be provided according to the different types of surgeries to facilitate the position usage of the instrument. For example, the positioning base may be a counterbore, a hole, a hemispherical body protruding from the positioning block. When the surgical instrument is a probe with a sharp tip, the positioning base may be matched with a counterbore; when the surgical instrument is a long-type drill surgical instrument, the positioning base may be matched with a hole; and when the surgical instrument is a suction device similar to a drainage device, the positioning base may be matched with the hemispherical positioning base. Therefore, the handpiece register of the present application may be more widely used in various surgeries.

By capturing the position of the light measurement unit, it can clearly know the position information of the optical measurement units and the front end of the instrument. Therefore, the system can still feedback the position information to the doctor to provide a reference for the doctor even if the naked eyes cannot judge the position of the front end of the instrument, so as to successfully complete the surgery.

By means of the above technical means, the present invention can indeed solve the problems in the prior art, and reduce and prevent the risk of surgical deviation or failure because of the misjudgment of position in the surgery.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view of a handpiece register according to a main example of the present invention;

FIG. 2 is a schematic view of using the handpiece register according to the main example of the present invention;

FIG. 3A to 3B are schematic views of plate bodies of the handpiece register according to possible embodiments of the present invention;

FIG. 4A to 4D are schematic views of positioning blocks with different positioning bases of the handpiece register according to the present invention.

### DETAILED DESCRIPTION OF THE PREFERRED EMBODIMENTS

In the following paragraphs cooperating with the diagrams; the embodiments of the handpiece register provided by the present invention will be further described.

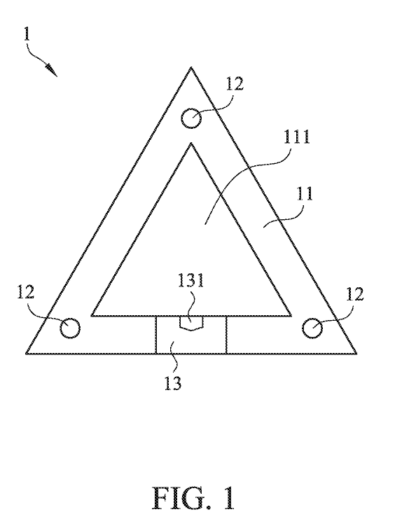

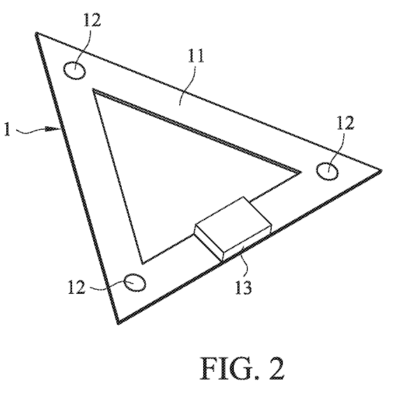

Please refer to FIG. 1 and FIG. 2, FIG. 1 is a schematic view of a handpiece register according to a main example of the present invention; and FIG. 2 is a schematic view of using the handpiece register according to the main example of the present invention.

The present invention is to provide a handpiece register, and more particularly to the handpiece register for positioning a instrument in a medical surgery. The handpiece register 1 comprises a plate body 11, more than three optical measurement units 12 and a positioning block 13.

In this embodiment, the plate body 11 is a flat triangular plate body, and the three optical measurement units 12 are respectively disposed on the same plane of the plate body 11. The positioning block 13 is also disposed on the plate body 11, and the positioning block 13 and the three optical measurement units 12 are disposed on the same plane. The positioning base 131 is disposed on the positioning block 13, and the positioning base 131 is used for abutting a front end of the instrument 2 which is provided for positioning.

Further, the plate body 11 may be a trigon, a polygon or a regular trigon, a regular polygon, and the three optical measurement units 12 are disposed on corners. Furthermore, a hollow portion 111 is disposed on the plate body 11 and relatively positioned above the positioning block 13. When the front end of the instrument is abutted on the positioning base 131, the accuracy of the position is not affected by abutting or against the plate body.

Furthermore, the positioning block 13 is connected to the plate body 11 by a pivoting method, so that the positioning block 13 may be rotated relative to the plate body 11; therefore, the positioning base 13 is rotated toward the hollow portion 111. In particular, when a plurality of positioning bases 131 are disposed on the positioning block 13, the corresponding positioning bases 131 may be turned to face the hollow portion 111 through a rotation.

Before the surgery, a plurality of optical measurement units are disposed on one part of the instrument (not shown), and the optical measurement units may be disposed on the instrument in advance or adhere to the instrument before the surgery. And the front end of the instrument with the optical measurement units are abutted to the positioning base 131 of the positioning block 13 of the handpiece register 1 provided by the present invention.

When performing a surgery, the handpiece register of the present invention is matched with an optical position detection system and a patient's 3D images, and the size of the instrument and the data of the light measuring units 12 are stored in the optical position detection system. After the front end of the instrument 2 is inserted into or abutted to the corresponding fixing seat 131, the optical position detection system records and calculates according to the measured relative position of a light sensing unit 12 to obtain the position of the front end of the instrument, and the optical position detection system simulates to display on the screen.

Additionally, the optical measurement units 12 may be active light emitting components or light reflecting components. The detection system is similar to an image capture device, which captures the instantaneous position data of the front end of the instrument by capturing the position of the light spot formed by the optical measurement units 12, and immediately displays the position information on the screen to the doctor during the surgery. Therefore, the reference data is provided to the doctor, and the necessary adjustments is made by the doctor to reduce and prevent surgical deviation or failure risk because of misjudgment of the position.

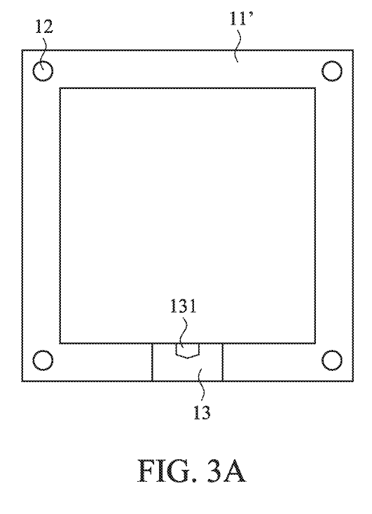

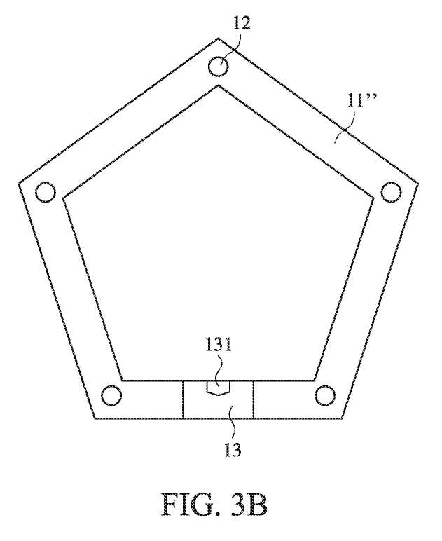

Additionally, please refer to FIG. 3A to FIG. 3B, FIG. 3A to 3B are schematic views of plate bodies of the handpiece register according to possible embodiments of the present invention.

The plate body 11 of the handpiece register 1 of the present invention is not only a triangle plat body, but also a tetragon plat body 11′ as shown in FIG. 3A, a pentagon plat body 11″ as shown in FIG. 3B, a polygon plat body or a regular polygon plate body. In order to achieve more accurate data feedback, the optical measurement units 12 may be disposed at the corners of the plate bodies 11′, 11″ according to the plate body 11 presented the number of edges of the polygons, and the positioning block 13 may be disposed between any two of the optical measurement units 12.

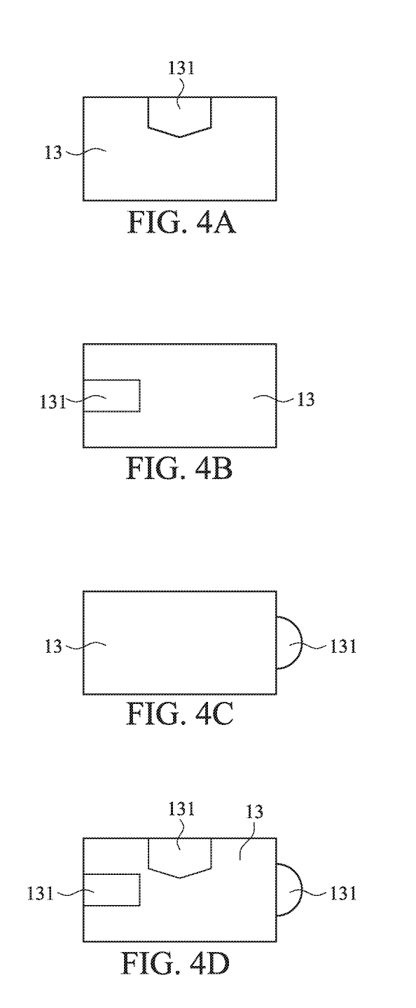

Additionally, please refer to FIG. 4A to FIG. 4D, FIG. 4A to 4D are schematic views of positioning blocks with different positioning bases of the handpiece register according to the present invention.

The positioning block 13 of the handpiece register 1 of the present invention may be a removable replacement component. As described above, the positioning block 13 is used for abutting the front end of the instrument which is provided for positioning. In order to treat different surgical procedures, an end port of the instrument will also be different, so the present invention also provides different aspects of the positioning base 131. As shown in FIG. 4A to FIG. 4D, the positioning base 13 may be a counterbore, a hole or a hemispherical body protruding from the positioning block. When the surgical instrument is a probe with a sharp tip, the positioning base may be matched with a counterbore as shown in FIG. 4A. When the probe is abutted on the bottom end of the counterbore, there is a fixing point is formed; therefore, the relative positional relationship between the optical measurement units on the probe and optical measurement units 12 on the board body 11 may be calculated by the software, and the shape and position of the instrument can be calculated accordingly. Identically, when the surgical instrument is a long-type drill surgical instrument, the positioning base may be matched with a hole as shown in FIG. 4B. After the drill is inserted in the hole and abutted to the bottom end of the hole, the position of the drill may be calculated by the software. Identically, when the surgical instrument is a suction device similar to a drainage device, the positioning base may be matched with the hemispherical positioning base as shown in FIG. 4C. When the suction device is abutted to the hemispherical positioning base, a central axis of the suction device may be directed to a hemisphere center of the hemispherical positioning base; therefore, the position of an end of the suction device may be calculated. Further, a plurality of positioning bases may also be disposed on the positioning blocks 13 as shown in FIG. 4D to treat the need for using a variety of instruments in the surgery. Therefore, the handpiece register 1 of the present invention may be more widely used in various operations.

I claim:

1. A handpiece register for positioning an instrument in a medical surgery, comprising:
a plate body;
more than three optical measurement units all disposed on the plate body; and
a positioning block disposed on the plate body, in which the positioning block and the three optical measurement units are located on the same plane of the plate body; and at least one positioning base disposed on the positioning block and used for abutting a front end of the instrument.

2. The handpiece register mentioned in claim 1, wherein at least three the optical measurement units are arranged in a triangle or a regular triangle or a regular polygon.

3. The handpiece register mentioned in claim 1, wherein the positioning block is disposed between any two of the optical measurement units.

4. The handpiece register mentioned in claim 1, wherein the plate body is a polygonal plate body with more than three sides, and at least one optical measurement unit is disposed at every corner of the plate body.

5. The handpiece register mentioned in claim 1, wherein the optical measurement units are light reflecting components.

6. The handpiece register mentioned in claim 1, wherein the optical measurement units are active light emitting components.

7. The handpiece register mentioned in claim 1, wherein the positioning block is a removable replacement component.

8. The handpiece register mentioned in claim 1, wherein the positioning base is a counterbore or a hole and is inserted in the positioning block, or the positioning base is a hemispherical body protruding from the positioning block.

9. The handpiece register mentioned in claim 1, wherein the plate body has a hollow portion located above the positioning block.

10. The handpiece register mentioned in claim 1, wherein the positioning block is pivoted on the plate body to rotate relative to the plate body, and more than two kinds of the positioning bases are disposed on the positioning block.
